(19) United States
(12) Patent Application Publication
(10) Pub. No.: US 2009/0018545 A1
(43) Pub. Date: Jan. 15, 2009

(54) Automatic Bone Traction Device

(76) Inventors: LI WU ZHENG, Hong Kong (CN); LIM KWONG CHEUNG, Hong Kong (CN); CHUNG MING WONG, Hong Kong (CN); KWOK HONG TONG, Hong Kong (CN)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 12/165,139

(22) Filed: Jun. 30, 2008

Related U.S. Application Data
(60) Provisional application No. 60/948,746, filed on Jul. 10, 2007.

(51) Int. Cl. A61B 17/58; A61H 7/00

(52) U.S. Cl. 606/90; 601/84

(57) ABSTRACT

An automatic bone traction device for correction bone deformities is disclosed. The automatic bone traction device can include a main body, a step motor, a series of transmission gears, and connectors. In one implementation, the step motor operates at a speed of eight steps per second, and the series of transmission gears can step down the motion of the step motor to two revolutions per day. The generated driving torque is then transmitted to a bone distractor, such as a commercially available intermittent distractor. The automatic bone traction device is capable of activating the bone distractor continuously at a rate of two revolutions per day. Advantageously, a flexible shaft is used to connect between the transmission gear assembly and the bone distractor. The flexible shaft allows the main body of the bone traction device to be mounted away from the bone distractor at a convenient location for the user.

### CROSS-REFERENCE TO A RELATED APPLICATION

This application claims the benefit of U.S. provisional application Ser. No. 60/948,746, filed Jul. 10, 2007, which is herein incorporated by reference in its entirety.

### FIELD OF THE INVENTION

The present invention is directed to a medical device for the correction of bone deformities and defects in maxillofacial and orthopedic surgery.

### BACKGROUND OF THE INVENTION

Distraction osteogenesis is a surgical method of generating new bone directly from divided bone segments by a mechanical device (distractor) producing controlled mechanical traction. In distraction osteogenesis, two important variables for the quality of newly formed bone are the rate and rhythm of distraction. The rate of distraction is the amount of bone lengthening in millimeters/day. The rhythm of distraction refers to how many steps each day the distractor is turned. Distraction osteogenesis developed from orthopedic surgery and has become a widely-accepted approach in the treatment of severe craniofacial deformities in the recent few years. The current distractors used in practice are activated manually and can only produce advancement once or twice a day, typically at a rate of 1 min/day until the desired endpoint is reached. This causes inconvenience and possible discomfort to the patients.

In the recent few years, attempts to achieve continuous advancement using an auto-drive system (motor or micro-hydraulic cylinder) have been reported. Compared with the traditional distractor which performs intermittent advancement once or twice per day, the automatic bone traction device can provide a very high rate of distraction. Continuous distraction is less painful and more convenient to patients, and has been found to be superior when compared with intermittent activation on bone regeneration. The disadvantages of the currently reported auto-drive system are that it is heavy, expensive and complicated to use. There is no auto-drive distractor available which can produce continuous distraction. A reliable, cost effective and easy to use automatic useful distraction system would be desirable and useful.

### BRIEF SUMMARY

The subject invention provides an automatic bone traction device. The automatic bone traction device can be provided comprising a main body, a flexible shaft and connectors. It is designed to be capable of attaching on a distractor and activating the distraction continuously at a fixed rate. The fixed rate can be two revolutions per day. The subject automatic bone traction device also has the potential to be attached on commercially available intermittent distractors for clinical use to achieve a continuous distraction. In a particular implementation, the distraction system uses a battery as the power source to drive a step motor moving with a frequency of, for example, eight steps per second. The driving torque is transmitted to the flexible shaft at the output end through a set of gears which step down the speed to two revolutions per day in a preferred embodiment.

Advantageously, the flexible shaft is used to connect between the transmission gear assembly and the bone distractor. The flexible shaft allows the main body of the bone traction device to be mounted away from the bone distractor at a convenient location for the user.

### BRIEF DESCRIPTION OF THE DRAWINGS

FIG. 1 shows an automatic bone traction device according to an embodiment of the subject invention.

FIG. 2 shows an upper housing of a main body of an automatic bone traction device according to an embodiment of the invention.

FIG. 3 shows a lower housing of a main body of an automatic bone traction device according to an embodiment of the invention.

FIG. 4 shows a battery cover in accordance with an embodiment of the invention.

FIG. 5 shows an hour shaft connected to a body containing movement mechanism for use in an automatic bone traction device according to an embodiment of the invention.

FIG. 6 shows a slider switch for an automatic bone traction device according to an embodiment of the invention.

FIG. 7 shows a PCB mount for an automatic bone traction device according to an embodiment of the invention.

FIG. 8 shows a mounting stud for an automatic bone traction device according to an embodiment of the invention.

FIG. 9 shows a connector for the flexible shaft for an automatic bone traction device according to an embodiment of the invention.

FIG. 10 shows a sleeve for insertion of a flexible shaft for an automatic bone traction device according to an embodiment of the invention.

FIG. 11 shows a connector for clamping to the flexible shaft for an automatic bone traction device according to an embodiment of the invention.

FIG. 12 shows a grip for holding a distractor in accordance with an embodiment of the invention.

FIG. 13 shows a pin that can be used for fitting a flexible shaft assembly to a main body of an automatic bone traction device according to an embodiment of the invention.

### DETAILED DESCRIPTION OF THE INVENTION

The subject invention pertains to an automatic bone traction device. According to embodiments of the subject invention, a torque generating mechanism such as a step motor and gear system can be used to continuously activate the distraction of a distractor. In addition, embodiments of the subject invention are capable of allowing user-convenient mounting of the automatic driving mechanism for the bone distractor. The power source is preferably electrical, alternating current or direct current, but can optionally be mechanical such as, for example, a spring.

The automatic bone traction device according to an embodiment can include a main body providing housing for the gear system and a flexible shaft for attaching to the distractor and activating the distraction of the distractor by transmitting the driving torque from the gear system to the distractor.

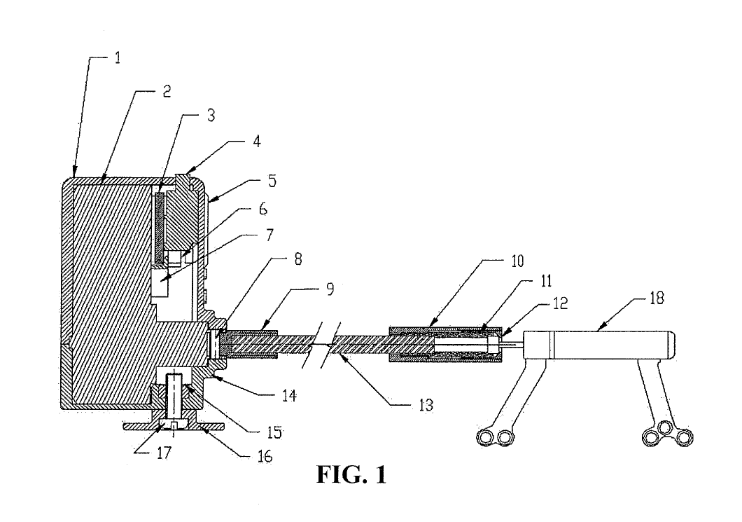

Referring to FIG. 1, a driving system according to an embodiment of the present invention can be coupled to a distractor 18. The distractor 18 can be any distractor suitable for performing osteogenesis.

Distraction osteogenesis is a surgical method of generating new bone directly from divided bone segments by controlled mechanical traction using a mechanical device—for example, distractor 18. The current distractors used in clinics are activated manually and can only produce intermittent advancement once or twice a day. This may cause inconvenience and discomfort to the patients.

The automatic bone traction device of a specific implementation of the present invention is designed to be capable of activating the distractor 18 continuously at a fixed rate of 2 revolutions per day in fine steps.

This automatic bone traction device can use one 1.5 V button battery 6 as the power source. The step motor inside the automatic bone traction device can be designed to move with a frequency of 8 steps per second under the control of circuitry such as a high torque clock movement mechanism 2. The driving torque of the step motor is transmitted to the flexible shaft 13 at the output end through a set of gears of the movement mechanism, which step down the speed to 2 revolutions per day. Hence, in this embodiment there are 345,600 of advancements for 1 revolution.

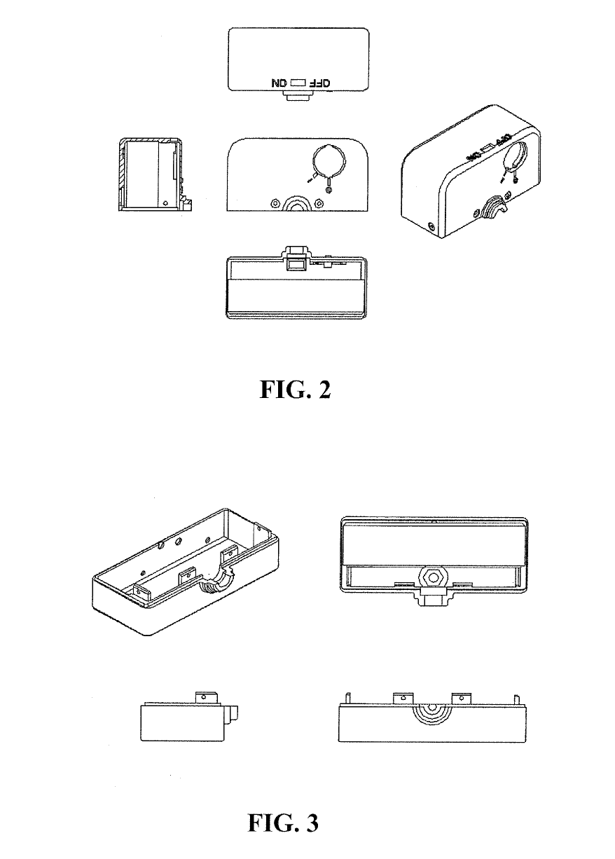

The automatic bone traction device comprises a main body, the flexible shaft 13 and the connectors as shown in FIG. 1. FIGS. 2-13 illustrate specific components of one embodiment of the subject apparatus. According to this embodiment, the main body includes the upper housing 1 as shown in FIG. 2 and the lower housing 14 as shown in FIG. 3, with the main core of the automatic bone traction device inside. Both upper housing and the lower housing can be made of durable acrylonitrile butadiene styrene (ABS) plastic. The upper housing and the lower housing can be fixed together with four self-tapping screws when assembled.

A semi-circular hole can be provided at the back of the top housing 1 that allows the operator of the system to check the rotational motion of the gearing system inside the movement mechanism, and thereby to check the strength of the 1.5V button battery 6.

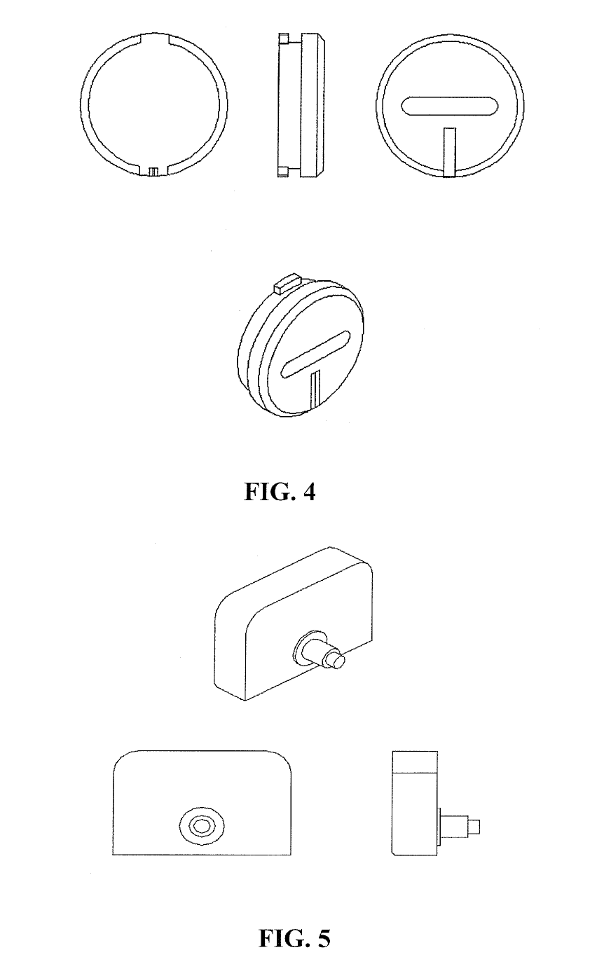

The 1.5V button battery can be removed from the main body by turning the battery cover 5, as shown in FIG. 4, counterclockwise with a flat-headed screwdriver or a coin. Thus it is rather easy to replace the used battery from the device. The battery cover 5 can be mounted onto the upper housing 1 after battery 6 replacement by aligning the vertical mark of the battery cover 5 to the vertical mark on the upper housing 1. For example, the battery cover 5 is turned clockwise until the vertical mark on the battery cover 5 aligns with the “I” mark on the upper housing.

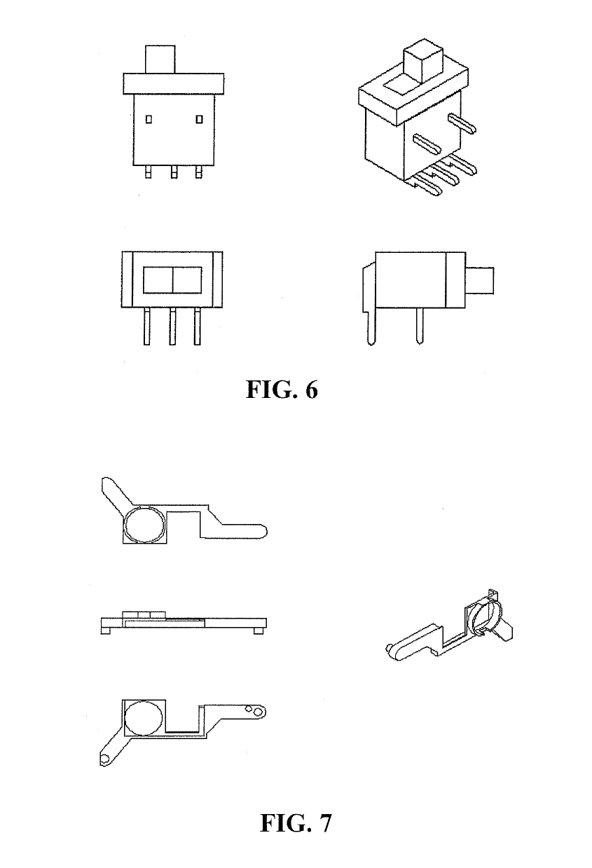

Inside the housing can be one high torque movement mechanism 2, the button battery 6, a slider-switch 4, PCB 3 and the PCB mount 7. In a specific embodiment, the high torque movement can be an AA battery powered SEIKO quartz high torque movement. The battery compartment of the “Seiko” brand high torque movement 2 for AA the battery is removed and the length of the minute axle and the second axle of the Seiko brand mechanism are shortened in order to leave room for attaching the flexible shaft 13 to the hour shaft of the movement mechanism as shown in FIG. 5. Then two pieces of electrical wire are connected internally in the movement mechanism and the other ends of the two wires are connected to the PCB 3 which holds the slider switch 4, as shown in FIG. 6, and the button battery 6.

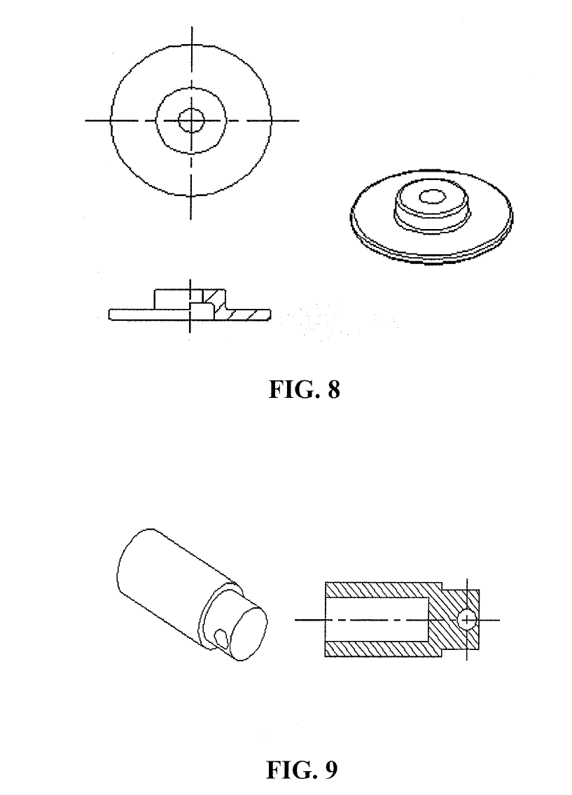

The PCB mount 7 which can be made of, for example, nylon is shown in FIG. 7. The PCB mount 7 is attached to the face of the SEIKO high torque movement 2 with glue. Then the assembly of the PCB 3 is inserted into the slot between the PCB mount 7 and the SEIKO high torque movement 2. The whole assembly is then housed into the upper housing 1. The stud 16 as shown in FIG. 8 is provided for mounting of the main body to the patient. It is fastened to the lower housing 14 with stainless steel nut and screw as shown in FIG. 1. The stud 16 can be made of ABS plastic material similar to the upper housing 1 and the lower housing 14. The central boss of the stud 16 allows the operator of the main body of the automatic bone traction device to attach it to the clothing or jacket of the patient. Further, the circular shape of the stud 16 permits the main body some degree of freedom when the device is in use.

The stainless steel flexible shaft 13 with connectors 9 and 11 at both ends is the bridge between the main body of the automatic bone traction device and the distractor 18. In one assembly method, one end of the flexible shaft can be inserted into the stainless steel connector 9 as shown in FIG. 9. Then, a large clamping force can be applied at the region of connection between the connector 9 and the flexible shaft 13. Physical deformation of the connector 9 will strongly grip the flexible shaft 13 to form an integral part. Hence, positive power transmission can be achieved.

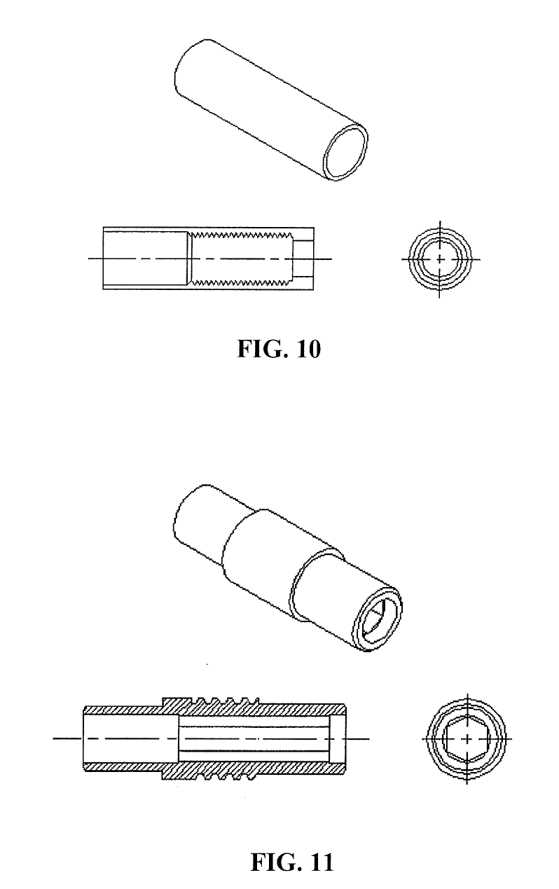

The other end of the flexible shaft 13 can be placed through the central hole of the stainless steel sleeve 10 as shown in FIG. 10 with the capped end nearer to the main body of the device. Then the stainless steel connector 11, as shown in FIG. 11, can be clamped with similar procedures as fixing the connector 9 onto the flexible shaft 13.

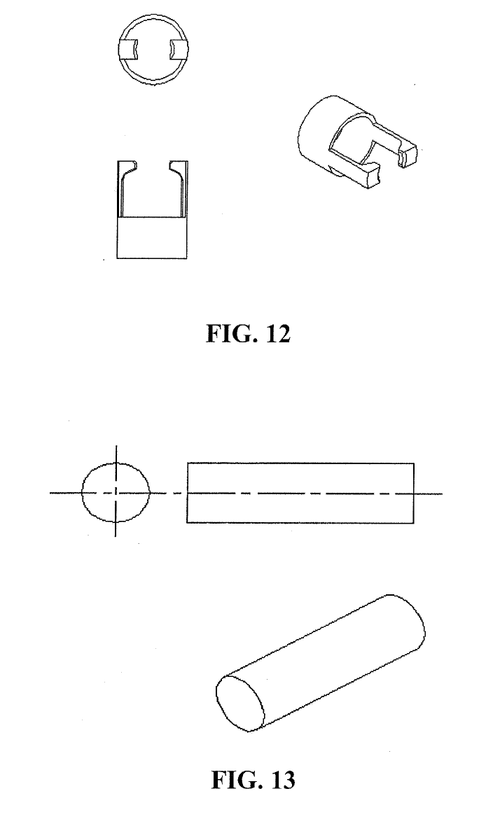

In an embodiment, the stainless steel grip 12 as shown in FIG. 12 is used for holding the distractor 18 when distraction is in progress. The two jaws of the grip 12 are retained in position by the stainless steel sleeve 10. The sleeve 10 can be retracted by turning it in clockwise direction. The two jaws of the grip will spring back when the sleeve 10 is retracted, thereby providing a means for easy removal of the distractor 18. The grip 12 can be fixed onto the connector 11 by laser welding.

The flexible shaft assembly can then fit to the hour shaft of the main body by inserting the pin 8 as shown in FIG. 13 through the hole at the rear of the flexible shaft assembly as shown in FIG. 1. After that, the main body of the automatic bone traction device and the flexible shaft assembly can be assembled into the upper housing 1 and the lower housing 14.

The button battery 6 can be inserted into the battery compartment and then the battery cover 5 is closed. The device can be turned on by using the slider switch 4 protruding at the top of the upper housing 1.

The step motor of the movement mechanism can turn eight steps per second and is controlled by the frequency of the movement circuit, which gives the auto-drive unit the possibility to change speed in a later stage of the development. The motion of the step motor is then stepped down through a series of gears connected to the drive shaft (for example, the hour shaft of the movement mechanism which moves at 2 revolutions per day in the present prototype example). The speed of the drive shaft can be adjusted or stopped by the switch system.

For an exemplary embodiment, the driving unit is provided in a system housing having a size of 5.5 cm×4.0 cm×2.8 cm and with a total weight of 55 g. The maximum force generated by the drive portion for the exemplary embodiment is 19N. Being different from the expensive and complicated drive system developed by other groups, this distraction system is driven by a step motor and gear system (similar to the core of a clock), which is more accurate in function and user-friendly. The driving unit system can also be easily attached on any commercial available intermittent distractors to achieve a continuous distraction.

The main body of the exemplary embodiment consists of the upper housing and the lower housing. Insides the housing is the core of the drive unit—distraction gears which include one 1.5 V dc step motor together with a gearing system. Following the similar approach as the core of a clock, a clock motor and gear system is utilized. The speed of the step motor is set at eight steps per second and is controlled by the frequency of the circuit, which gives the auto-drive unit the possibility to change speed in the later stage of the development. The motion of the step motor is then stepped down through a series of gears to the driving shaft, which is designed to move at two revolutions per day at present. The speed of the drive shaft can then be adjusted or stopped by a switch system and the whole system is then powered by one button battery. The circuit board together with the switch system and the button battery are mounted on the outside of the gearing system by the PCB mount. A removable battery cover which allows easy reloading of battery can be found on the front panel of the upper housing. One circular plastic stud is fixed on the bottom of the lower housing. The stud allows the auto-drive unit to be mounted onto the patient easily and allows the unit to rotate to any desired orientation.

The flexible shaft conveys torque from the drive shaft to the distractor, and can be made of any suitable durable material such as stainless steel or nitinol, for example. In the exemplary embodiment, a stainless steel flexible cord is connected to the drive shaft of the main body by a pin which passes through the output shaft and the connector. This stainless steel flexible cord connects the driving shaft of the electronic module in the main body to the distractor. The connector is clamped on one end of the flexible cord to ensure positive transmission of the torque to the other end of the cord. On the other end of the flexible cord is a mounting mechanism which consists of a jaw, a hexagon socket and an outer sleeve. The whole mechanism can be used to clamp the handle of the distractor during the distraction period. When the distraction period is over, the sleeve can be turned anticlockwise to allow the spring back of the jaw. Then the distractor and the drive unit can be separated and the drive unit can be detached from the patient.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

It should be understood that the examples and embodiments described herein are for illustrative purposes only and that various modifications or changes in light thereof will be suggested to persons skilled in the art and are to be included within the spirit and purview of this application.

We claim:

1. An automatic bone traction device, comprising:
a main body comprising a torque mechanism capable of continuously providing torque; and
a flexible shaft connected to the main body for transmitting the torque from the torque mechanism to a distractor.

2. The automatic bone traction device according to claim 1, wherein the torque mechanism is electrically powered.

3. The automatic bone traction device according to claim 1, wherein the torque mechanism comprises:
a step motor providing an initial output torque; and
a plurality of gears to step down the initial output torque before transmitting the torque.

4. The automatic bone traction device according to claim 3, wherein the step motor revolves at a speed of about eight steps per second and the gears step down the motor speed to about two steps per second.

5. The automatic bone traction device according to claim 3, further comprising:
a power source within the main body for driving the step motor.

6. The automatic bone traction device according to claim 5, wherein the power source comprises a battery.

7. The bone traction device according to claim 1, wherein the torque mechanism comprises a high torque clock movement.

8. The bone traction device according to claim 7, wherein the high torque clock movement comprises an hour shaft, and wherein the flexible shaft is fitted to the hour shaft of the high torque clock movement.

9. The automatic bone traction device according to claim 1, wherein the distractor is an intermittent orthopedic distractor.

10. The automatic bone traction device according to claim 1, wherein the flexible shaft comprises a stainless steel flexible cord.

11. The automatic bone traction device according to claim 1, wherein the flexible shaft is releasably detachable from the distractor.

12. The automatic bone traction device according to claim 11, further comprising:
a connector joining one end of the flexible shaft to the main body; and
a connector for releasably joining the other end of the flexible shaft to the distractor.

13. The automatic bone traction device according to claim 12, wherein the connector for releasably joining the other end of the flexible shaft to the distractor comprises a double action detachable mechanism.

14. The automatic bone traction device according to claim 13, wherein the double action detachable mechanism comprises:
a retractable sleeve; and
a stainless steel grip comprising jaws, wherein the jaws are capable of being retained in a gripping position by the retractable sleeve and spring open when the retractable sleeve is retracted.

15. The automatic bone traction device according to claim 1, wherein the main body further comprises:
a switch system for adjusting and stopping the speed of the torque mechanism.
